(19) United States
(12) Patent Application Publication
(10) Pub. No.: US 2026/0242817 A1
(43) Pub. Date: Aug. 20, 2026

(54) OPTIMIZED MAIZE CODING SEQUENCES

(71) Applicant: INARI AGRICULTURE TECHNOLOGY, INC., Cambridge, MA (US)

(72) Inventors: Ruijuan LI, Belmont, MA (US); Malachy CAMPBELL, Randolph, MA (US); Karl KREMLING, Somerville, MA (US)

(21) Appl. No.: 19/415,370

(22) Filed: Dec. 10, 2025

Related U.S. Application Data
(60) Provisional application No. 63/734,643, filed on Dec. 16, 2024.

(51) Int. Cl. C12N 15/82; C07K 14/415; C12N 9/22; C12N 9/78; C12N 15/11; C12Q 1/6869; C12Q 1/6895

(52) U.S. Cl. CPC C12N 15/8282; C07K 14/415; C12N 9/226; C12N 9/78; C12N 15/111; C12N 15/8201; C12N 15/8275; C12Q 1/6869; C12Q 1/6895; C12N 2310/20; C12Q 2600/13; C12Y 305/04001; C12Y 305/04002

(57) ABSTRACT

This disclosure pertains to the use of ancestral amino acid polymorphisms in maize plant germplasm to enhance agronomic traits. Specifically, it covers methods for identifying these polymorphisms, characterizing their impacts, and employing them in breeding strategies such as marker-assisted and genomic selection. The use of these polymorphisms can lead to the development of maize varieties with superior yield, stress tolerance, and nutritional content, addressing both agricultural challenges and food security concerns.

### CROSS-REFERENCE TO RELATED APPLICATIONS

This application claims the benefit of Provisional Application U.S. Ser. No. 63/734,643 filed on Dec. 16, 2024, all of which including the sequence listing and large table is herein incorporated by reference in its entirety.

### REFERENCE TO SEQUENCE LISTING SUBMITTED ELECTRONICALLY

The instant application contains a Sequence Listing which has been submitted electronically in XML format and is herein incorporated by reference in its entirety. Said XML copy, created on Dec. 8, 2025, is named “P14652US01.xml” and is 55,865,072 bytes in size.

### LARGE TABLE

The instant application includes a Large Table named “Table1.txt” (Size: 17,328,770 bytes and Date of Creation: May 15, 2024), which is submitted in electronic form in ASCII plain text format via the United States Patent and Trademark Office (USPTO) patent electronic filing system and is hereby incorporated by reference in its entirety.

LENGTHY TABLES
The patent application contains a lengthy table section. A copy of the table is available in electronic form from the USPTO web site (https://seqdata.uspto.gov/docdetail?docId=US20260242817A1). An electronic copy of the table will also be available from the USPTO upon request and payment of the fee set forth in 37 CFR 1.19(b)(3).

### FIELD

The disclosure is generally related to the identification and use of ancestral amino acid polymorphisms in maize plant germplasm.

### BACKGROUND

The cultivation and genetic improvement of maize (Zea mays) have vast implications for agriculture, particularly in enhancing crop yield, adaptability, and resilience to environmental stressors. Traditional breeding techniques, although effective, are time-consuming and often result in limited genetic diversity within the cultivated germplasm. Advances in molecular biology and genetics have enabled the identification and utilization of specific genetic polymorphisms that can predict desirable traits, thus accelerating the breeding process.

### SUMMARY

This disclosure relates to the identification, characterization, and use of ancestral amino acid polymorphisms in the germplasm of maize plants to improve agronomic fitness and/or enhance various desirable agronomic traits. Specifically, the disclosure includes methods for selecting maize plants that possess one or more beneficial ancestral amino acid polymorphisms associated with improved agronomic fitness, quality and/or nutritional traits. In certain embodiments, such improved agronomic fitness can include traits such as increased yield, improved abiotic stress tolerance and/or biotic stress tolerance in comparison to control maize plants lacking the ancestral amino acid polymorphisms.

Methods are provided for producing a maize plant with nutritional and quality traits for human consumption and for use as animal feed. In some embodiments, such improved nutritional quality includes increased provitamin A content, as achieved through biofortified maize varieties like golden maize, increased lysine and/or tryptophan content, or higher iron and zinc concentrations for addressing micronutrient deficiencies.

In other embodiments, the improved nutritional quality comprises reduced phytate content to enhance mineral bioavailability, optimized starch composition with higher amylopectin levels, or increased oleic acid content for healthier oil with extended shelf life. These traits contribute to both the nutritional and functional value of maize.

Further embodiments include nutritional traits such as elevated anthocyanin levels for enhanced antioxidant properties or improved dietary fiber content to support digestive health. These traits are developed through advanced breeding techniques or biotechnological interventions to meet specific dietary and health needs.

Still further embodiments provide maize plants with high folate content for maternal and neonatal health, or increased tocopherol (vitamin E) levels to support immune function and provide antioxidant benefits. Such innovations in maize contribute to addressing global nutritional challenges and improving the quality of staple foods.

By utilizing these polymorphisms, it is possible to provide maize varieties with superior agronomic performance, addressing both agricultural and nutritional needs.

Methods are provided for improving maize plant germplasm comprising: (i) contacting the genomes of a plurality of maize plant cells comprising one or more codons encoding a derived amino acid polymorphism in at least one target maize gene with one or more gene editing reagents capable of converting said codons encoding the derived amino acid polymorphism to one or more codons encoding a corresponding ancestral amino acid polymorphism of the target maize gene;

and (ii) selecting a maize plant cell or plant wherein at least one codon encoding one or more derived amino acid polymorphisms of at least one target maize gene has been converted to the codon encoding the corresponding ancestral amino acid polymorphism of the target maize gene to obtain a first selected plant cell or plant containing the converted codon. Selected maize plants or parts thereof produced by the aforementioned methods are also provided.

Methods are provided for improving maize plant germplasm comprising: (i) selecting a first parental maize plant comprising one or more codons encoding at least one desired ancestral amino acid polymorphism in one of more first parental plant gene(s) and a second parental maize plant comprising one or more codons encoding at least one desired ancestral amino acid polymorphism in one of more second parental plant gene(s); (ii) crossing the first and second parental maize plants to obtain a population of progeny plants; (iii) screening the population of progeny plants for a progeny plant comprising a combination of desired ancestral amino acid polymorphisms in both the first and second parental plant gene(s); and (iv) isolating a progeny maize plant comprising the combination of desired ancestral amino acid polymorphisms from the population of maize plants. Isolated maize plants or parts thereof produced by the aforementioned methods are also provided.

Methods are provided for improving maize plant germplasm comprising: (i) detecting a maize plant comprising a conversion of at least one codon encoding a derived amino acid polymorphism in at least one target gene to a codon encoding a corresponding ancestral amino acid polymorphism in a population of maize plants comprising individual maize plants comprising the codon encoding the derived amino acid polymorphism or the ancestral amino acid polymorphism; and (ii) isolating the maize plant comprising the codon encoding the ancestral amino acid polymorphism from the population of maize plants. Isolated maize plants or parts thereof produced by the aforementioned methods are also provided.

Methods are provided for identifying or selecting maize plants with improved fitness comprising: (i) identifying at least one ancestral amino acid polymorphism by performing genome-wide multiple sequence alignment between maize germplasm and related grass species, wherein the ancestral amino acid polymorphism comprises the allele found in at least 70% of the related grass species; (ii) identifying at least one derived amino acid polymorphism of the ancestral amino acid polymorphism, wherein a protein comprising the derived amino acid polymorphism has a predicted protein stability which is increased or decreased in comparison to a protein comprising the ancestral amino acid polymorphism; and optionally (iii) selecting a maize plant comprising the ancestral amino acid polymorphism from a population of maize plants comprising individual maize plants comprising the derived amino acid polymorphism or the ancestral amino acid polymorphism. Selected maize plants or parts thereof produced by the aforementioned methods are also provided.

Also provided are methods of identifying or selecting maize plants with improved agronomic fitness comprising: (i) identifying at least one ancestral amino acid polymorphism by performing genome-wide multiple sequence alignment between maize germplasm and related grass species, wherein the ancestral amino acid polymorphism comprises the amino acid polymorphism found in at least 70% of the related grass species; (ii) identifying at least one derived amino acid polymorphism of the ancestral amino acid polymorphism, wherein a protein comprising the derived amino acid polymorphism has a predicted protein stability which is increased or decreased in comparison to a protein comprising the ancestral amino acid polymorphism; and optionally (iii) selecting a maize plant comprising the ancestral amino acid polymorphism from a population of maize plants comprising individual maize plants comprising the derived amino acid polymorphism or the ancestral amino acid polymorphism. Selected maize plants or parts thereof produced by the aforementioned methods are also provided.

Also provided are methods for producing a maize plant comprising in its genome at least one introgressed maize gene comprising a codon encoding an ancestral amino acid polymorphism, comprising the steps of: (a) crossing a first maize plant with a second maize plant, wherein the second maize plant comprises an ancestral amino acid polymorphism, to obtain a population segregating for the ancestral amino acid polymorphism; (b) selecting a seed or progeny maize plant from step (b) by its genotype, wherein the selected seed or progeny maize plant comprises a genotype comprising an ancestral amino acid polymorphism, thereby obtaining a selected maize plant comprising in its genome at least one introgressed maize gene comprising a codon encoding an ancestral amino acid polymorphism. Selected maize plants or parts thereof produced by the aforementioned methods that comprise the introgressed maize gene comprising the codon encoding ancestral amino acid polymorphism are also provided.

Also provided are maize plant cell comprising a substitution of one or more nucleotides in a codon of an endogenous gene encoding a derived amino acid polymorphism with one or more nucleotides to provide a codon variant encoding an ancestral amino acid polymorphism set forth in SEQ ID NO: 1-21,318, wherein one or more linked and/or unlinked DNA polymorphisms present in the maize plant cell comprising the codon variant are absent from maize plant cells comprising an endogenous maize gene which encodes the ancestral amino acid polymorphism. Also provided are maize plant cells comprising an insertion and/or deletion or substitution of one or more nucleotides in a codon of an endogenous gene encoding a derived amino acid polymorphism with one or more nucleotides to provide a codon variant encoding an ancestral amino acid polymorphism set forth in SEQ ID NO: 1-21,318, wherein one or more linked and/or unlinked DNA polymorphisms present in the maize plant cell comprising the codon variant are absent from maize plant cells comprising an endogenous maize gene which encodes the ancestral amino acid polymorphism.

Maize plant parts comprising any of the aforementioned maize plant cells, optionally wherein the part is a seed, are also provided.

### Definitions

As used herein, the term “allelic variants” is defined as proteins having at least 95%, 98%, or 99% sequence identity to SEQ ID NO: 1-21,318 wherein one of more amino acid residues other than the variant residues set forth in SEQ ID NO: 1-21,318 are either absent or substituted with a different amino acid and/or wherein one or more additional amino acid residues are inserted in SEQ ID NO: 1-21,318.

As used herein, the term “ancestral amino acid polymorphism” refers to an amino acid polymorphism that is observed at a given amino acid position in at least 70% or more of the orthologous proteins in multiple sequence alignment of the orthologous proteins multiple related species.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, “converted codon” refers to a nucleic acid sequence encoding an amino acid, wherein the encoded amino acid has been changed from a derived amino acid polymorphism to an ancestral amino acid polymorphism.

As used herein, the terms “correspond,” “corresponding,” and the like, when used in the context of an nucleotide position, mutation, and/or substitution in any given polynucleotide (e.g., an allelic variant of SEQ ID NO: 1) with respect to the reference polynucleotide sequence (e.g., SEQ ID NO: 1) all refer to the position of the nucleotide in the given sequence that has identity to the nucleotide in the reference nucleotide sequence when the given polynucleotide is aligned to the reference polynucleotide sequence using a pairwise alignment algorithm (e.g., CLUSTAL O 1.2.4 with default parameters).

As used herein, the term “derived amino acid polymorphism” refers to polymorphisms that occur in an orthologous gene in a phylogeny (i.e., a set of species derived through evolution from a common ancestor) and that differ from the ancestral amino acid polymorphism.

As used herein, the phrases “elite maize line” or “elite maize plant” refer to any line or plant which has undergone breeding to provide one or more trait improvements (e.g., desirable agronomic performance (typically commercial production) or superior grain quality). In some cases, an elite line can be an agronomically or otherwise superior line or variety that has resulted from several or many cycles of breeding and selection for one or more trait improvements (e.g., superior agronomic performance or superior grain quality). Similarly, “elite germplasm” is a germplasm resulting from breeding and selection for desirable agronomic performance (typically commercial production). Such germplasm can be agronomically superior germplasm, derived from and/or capable of giving rise to a plant with superior agronomic performance, such as an existing or newly developed elite line of soybean. Elite crop plant lines include plants which are an essentially homozygous, e.g., inbred or doubled haploid. Elite crop plants can include inbred lines used as is or used as pollen donors or pollen recipients in breeding (e.g., used to produce F1 plants). Elite crop plants can include inbred lines which are selfed to produce non-hybrid cultivars or varieties. Elite crop plants can include hybrid F1 progeny of a cross between two distinct elite inbred or doubled haploid plant lines.

As used herein, the phrase “endogenous gene” refers to the native form of a gene unit in its natural location in the genome of an organism.

As used herein, the term “expression” refers to the production of a functional end-product (e.g., an mRNA, guide RNA, or a protein) in either precursor or mature form.

As used herein, the terms “include,” “includes,” and “including” are to be construed as at least having the features to which they refer while not excluding any additional unspecified features.

The term “isolated” as used herein means having been removed from its natural environment.

As used herein, the term “introduced” means providing a nucleic acid (e.g., expression construct) or protein into a cell. Introduced includes reference to the incorporation of a nucleic acid into a eukaryotic or prokaryotic cell where the nucleic acid can be incorporated into the genome of the cell and includes reference to the transient provision of a nucleic acid or protein to the cell. Introduced includes reference to stable or transient transformation methods. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment can be incorporated into the genome of the cell (e.g., nuclear chromosome, plasmid, plastid, chloroplast, or mitochondrial DNA), converted into an autonomous replicon, or transiently expressed (e.g., transfected mRNA).

As used herein, a “non-natural” or “non-naturally occurring” mutation refers to a mutation in a gene which is generated via human intervention or descended from the mutation generated via human intervention. Non-limiting examples of human intervention which can be used to generate a non-naturally occurring mutation include mutagenesis (e.g., chemical mutagenesis, ionizing radiation mutagenesis), mutagenesis followed by DNA sequence-based screening and selection (TILLING), and targeted genetic modifications (e.g., CRISPR-based methods, TALEN-based methods, zinc finger-based methods).

As used herein, the phrase “orthologous genes” or term “orthologs” refer to genes in different species that originated from a common ancestor and were separated by a speciation event.

As used herein, the term “plant” includes a whole soybean plant and any descendant, cell, tissue, part, or parts of the plant. The term “plant” thus includes reference to an immature or mature whole soybean plant, including a plant from which seed or grain or anthers have been removed.

The term “plant part” include any part(s) of a plant, including, for example and without limitation: seed (including mature seed and immature seed); grain; stover; a plant cutting; a plant cell; a plant cell culture; or a plant organ (e.g., pollen, embryos, pods; flowers, fruits, shoots, leaves, roots, stems, and explants). A plant tissue or plant organ can be a seed, protoplast, callus, or any other group of plant cells that is organized into a structural or functional unit. A plant cell or tissue culture can be capable of regenerating a plant having the physiological and morphological characteristics of the plant from which the cell or tissue was obtained, and of regenerating a plant having substantially the same genotype as the plant. Regenerable cells in a plant cell or tissue culture can be embryos, protoplasts, meristematic cells, callus, pollen, leaves, anthers, roots, root tips, flowers, or stalks. In contrast, some plant cells are not capable of being regenerated to produce plants and are referred to herein as “non-regenerable” plant cells.

As used herein, the term “variety” refers to a group of similar plants that by one or more structural features, genetic features, and/or performance can be distinguished from other varieties within the same species. In certain embodiments, the term variety refers to the botanical taxonomic designation whereby variety is ranked below species or subspecies, as well as the legal definition whereby the term “variety” refers to a commercial plant that is protected under the terms outlined in the International Convention for the Protection of New Varieties of Plants.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### DETAILED DESCRIPTION

The limited genetic diversity and inability to pyramid variation from multiple lines in adjacent genetic regions due to linkage disequilibrium through breeding has left the maize breeding germplasm with many deleterious mutations. These alleles are likely to pose significant constraints on inbred and hybrid line development. The development of high yielding crop varieties through conventional breeding is dependent on selecting the highest performing individuals. Selection can be based on the overall phenotypic values of an individual or hybrid, their estimated genomic breeding values, or predicted genomic breeding values. It is the cumulative effect of all alleles at functional sites in the genome that drive some part of these metrics.

Deleterious alleles, particularly those that are weakly deleterious pose a significant challenge for breeding programs that are dependent on recurrent selection of genetic variation in populations to improve performance. Selection against a deleterious allele is proportional to the size of the deleterious effect. The ability to purge a deleterious allele from the population is highly dependent on whether that allele is in genetic linkage with a favorable allele of equal or greater effect on the trait of interest, the size of the breeding population, and the frequency of the favorable and unfavorable alleles at that position in the breeding population.

The effect of weakly deleterious alleles on a complex trait like yield is small, and so, their removal from a breeding population through selection and recombination is extremely difficult. If the cumulative effects from linked beneficial alleles in a genomic region exceeds that of the deleterious allele, then the effect of the deleterious allele will effectively be masked. Moreover, while the effect of each weakly deleterious allele can be small, their cumulative effects on inbred and hybrid performance can be substantial. If provided with sufficient information to identify functionally important sites for complex traits like yield and knowledge of the beneficial alleles at those sites, precise, multiplexed genome editing approaches can be leveraged to ‘correct’ these weakly deleterious alleles and overcome the limitation these alleles pose on genetic improvement.

In certain embodiments disclosed herein, methods of (1) identifying putatively functional sites (e.g., amino acid polymorphisms) from genetic variation in maize and related grass species (e.g., grass species in the andropogonodeae clade), (2) identifying alleles that are likely beneficial and deleterious at those sites, and (3) validating the cumulative effects of these alleles on inbred and/or hybrid performance are disclosed.

### Identification of Ancestral Amino Acid Polymorphisms

Certain embodiments of the methods disclosed herein involve identifying specific ancestral amino acid polymorphisms present in orthologous genes. Without seeking to be limited by theory it is believed that such ancestral amino acid polymorphisms which have been retained through evolutionary processes can contribute positively to agronomic fitness (e.g., traits including yield, abiotic stress tolerance, and/or biotic stress tolerance). Ancestral amino acid polymorphisms are identified using a combination of genomic sequencing and bioinformatics analysis, comparing orthologous proteins of related grass species (e.g., grass species in the andropogonodeae clade) to the orthologous protein(s) of modern maize cultivars. In one method, whole-genome alignments can be performed in 19 grass species (e.g., grass species in the andropogonodeae clade) related to maize. In one method, alignments were performed utilizing the methods in Wu, Y., Johnson, L., Song, B., Romay, C., Stitzer, M., Siepel, A., Buckler, E., & Scheben, A. (2022), A multiple alignment workflow shows the effect of repeat masking and parameter tuning on alignment in plants is disclosed in Plant Genome, 15, e20204; which can be adapted for this purpose and is hereby incorporated by reference in its entirety. In an embodiment, ancestral amino acid polymorphisms are inferred from a genome-wide multiple sequence alignment between orthologous genes in maize (e.g. B73 v4) and related grass species (e.g., species in the andropogonodeae clade). In certain embodiments, the ortholog in the maize genome is compared to at least 10, 12, 14, 16, 18, or 19 orthologs from at least 10, 12, 14, 16, 18, or 19 grass species. Ancestral amino acid polymorphisms can be called for each position in the maize ortholog (e.g. B73 v4) where a reliable alignment could be obtained. An amino acid polymorphism can be considered to be ancestral if it was found in 70% or more of the orthologous proteins with an unambiguous allele call at that position. In certain embodiments, derived amino acid polymorphisms at each site in an ortholog can be identified by comparing ancestral amino acid polymorphisms with alleles of the orthologous proteins called from more than 1300 diverse maize inbreds. Any allele that does not match the ancestral amino acid polymorphism can be considered a derived amino acid polymorphism. In certain embodiments, derived amino acid polymorphisms including those set forth in Table 1 and in SEQ ID NO: 1-21 can be targeted for conversion to ancestral amino acid polymorphisms.

### Characterization of Useful Ancestral Amino Acid Polymorphisms

In one embodiment, ancestral and derived amino acid polymorphisms are constructed from the corresponding gene models from a maize genomic sequence database (e.g., B73v4 or other maize genome on the world wide web internet site “maizegdb.org;” Woodhouse et al., BMC Plant Biol 21, 385. doi: 10.1186/s12870-021-03173-5). In certain embodiments, the longest gene model is selected for each gene in the selected maize genome (e.g., B73v4). Next, a determination is made as to whether the selected maize genome (e.g., B73v4) harbored the ancestral or derived amino acid polymorphism for each variant that overlapped with these regions. In embodiments, the determination is made by aligning the maize ortholog with orthologs from other grass species (e.g., species in the andropogonodeae clade). If the allele found in the selected maize genome (e.g., B73v4) is ancestral, the sequence can be translated in silico to generate an ancestral protein sequence, and the derived amino acid polymorphism can be substituted, and the corresponding sequence can be translated in silico to generate the derived protein sequence. If the allele found in the selected maize genome (e.g., B73v4) is derived, the sequence can be translated in silico to generate a derived protein sequence, and the ancestral amino acid polymorphism was substituted, and the corresponding sequence was translated in silico to generate the derived protein sequence.

Once identified, these polymorphisms are characterized to determine their impact on protein stability, protein function, and/or overall plant physiology. Assays can include enzyme activity measurements, protein stability tests (in vivo, in vitro, and/or in silico), and in planta evaluations under varying environmental conditions. In certain embodiments, a protein stability score is used to characterize ancestral and derived amino acid polymorphisms. In certain embodiments, protein stability scores can be assigned to orthologs containing ancestral or derived amino acid polymorphisms by adaptations of stability scoring methods disclosed by Rocklin et al (2017) Science. 2017 Jul. 14; 357(6347): 168-175., hereby incorporated by reference in its entirety for this purpose. In certain embodiments, a protein stability score is defined as the difference between the measured EC₅₀ (e.g., protease concentration at which half of the cells with a given ortholog with a given amino acid polymorphism resist protease cleavage of the ortholog) and the predicted EC₅₀ of the ortholog in the unfolded state can be utilized in the characterization of ancestral vs. derived amino acid polymorphisms. In certain embodiments, a computer model can be used to predict protein stability (e.g., provide a protein stability score). In certain embodiments, a protein stability score can be determined using a model disclosed in the aforementioned Rocklin et al (2017) publication. In a completely unfolded state, a protein's protease sensitivity is dependent on its potential cleavage sites. Rocklin et al. randomly scrambled ~18,000 sequences, which are likely to be in a completely unfolded state and performed yeast display and protease exposure assays. The Rocklin et al. observed EC₅₀ values were compared with the unfolded EC₅₀ values to determine protein stability scores. These data were used by Rocklin et al. to train a computer model to predict EC₅₀ values for any protein sequence in an unfolded state. In certain embodiments, the Rocklin et al. computer model available at the https internet site “github.com/asford/protease_experimental_analysis” or adaptations thereof can be used for assigning protein stability scores. Additional computer modeling tools that can be adapted for use in assigning protein stability scores to orthologs containing ancestral or derived amino acid polymorphisms include: FoldX (http internet site “foldxsuite.crg.eu/” and Buß et al., 2020, doi: 10.1016/.csbi.2018.01.002); Rosetta (https internet site “rosettacommons.org/software/” and Leman et al. 2021 doi: 10.1038/841592-020-0848-2); PremPS (https internet site lilab.jysw.suda.edu.cn/research/PremPS/, Chen et al. 2020 DOI: 10.1371/joumal.pcbi. 1008543); PRIME (https internet site “github.com n/Pro-Prime” and Jiang et al. 2024, doi: 10.1126/sciady.adr2641); AE Embedder (Boyer et al., 2023, doi: 10.48550/arXiv.2305.19801); RaSP (https internet site “github.com/dohlee/rasp-pytorch” and Blaabjerg et al. 2023, doi: 10.7554/eLife.82593); ACDC-NN-Seq (https internet site “pypi.org/project/acdc-nn/” and Pancotti et al. doi: 10.3390/genes 12060911); ThermoNet (https internet site “github.com/gersteinlab/ThermoNet” and Li et al., 2020, doi: 10.1371/journal.pcbi. 1008291); iStable (Chen et al., 2020, doi: 10.1016/j.csbj.2020.02.021); and LM-GVP (https internet site “github.com/aws-samples/lm-gvp” and Wang et al. 2022, doi: 10.1038/s41598-022-10775-y).

In certain embodiments, a protein function score is used to characterize ancestral and derived amino acid polymorphisms. Computer modeling tools that can be adapted for use in assigning protein function scores to orthologs containing ancestral or derived amino acid polymorphisms include: PROVEAN (http internet site “provean.jcvi.org/index_php” and Choi and Chan, 2015, doi: 10.1093/bioinformatics/bty195); Envision (Gray et al., 2018, doi: 10.1016/j.cels.2017.11.003); EVMutation (Hopf et al., 2017, doi: 10.1038/nbt.3769); Tranception (Notin et al. 2022, 10.48550/arXiv,2205.13760); SHINE (Fan et al., 2022, doi: 10.1093/bib/bbac584); CPT (Jagota et al., 2023, doi: 10.1186/s13059-023-03024-6); EvolMPNN (Zhong and Mottin, 2024, doi: 10.48550/arXiv.2402.13418); VariPred (Lin et al. 2024, doi: 10.1038/s41598-024-51489-7); VELM; gMVP (Zhang et al. 2023, doi: 10.1038/s42256-022-00561-w); AlphaMissense (Cheng et al. 2023, doi: 10.1126/science.ado749); and AlphaScore. In certain embodiments, maize plants or germplasm comprising at least one ancestral amino acid polymorphism associated with increased protein function as compared to a protein having the derived allele are selected. In certain embodiments, maize plants or germplasm comprising at least one ancestral amino acid polymorphism associated with decreased protein function as compared to a protein having the derived allele are selected. In certain embodiments, such protein function(s) which are increased or decreased can include enzymatic activity, transcription factor activity, and/or binding activity (e.g., to another protein or ligand).

### Gene Editing Techniques for Conversion of Alleles

Many methods of gene editing in maize can be used in certain embodiments to obtain converted codons. Targeted editing of nucleic acid sequences, for example, the targeted cleavage or the targeted introduction of a specific modification into genomic DNA, is a beneficial approach for the study of gene function and incorporation of desired agronomic traits into plants. An ideal nucleic acid editing technology possesses three characteristics: (1) high efficiency of installing the desired modification; (2) minimal off-target activity; and (3) the ability to be programmed to edit precisely any site in a given nucleic acid, e.g., any site within the plant genome. Current plant genome engineering tools, including engineered zinc finger nucleases (ZFNs), transcription activator like effector nucleases (TALENs), and the RNA-guided DNA endonuclease Cas9 and Cas12, effect sequence specific DNA cleavage in a genome. This programmable cleavage can result in mutation of the DNA at the cleavage site via non-homologous end joining (NHEJ) or replacement of the DNA surrounding the cleavage site via homology directed repair (HDR).

In some embodiments, utilization of genome editing tools enabled by clustered regularly interspaced short palindromic repeats (such as CRISPR Cas9 or Cas12-mediated homology directed repair, nucleobase base editing, and prime editing), candidate mutations can be made in a targeted fashion in plant cells, protoplast, and/or plantlets.

A variety of gene editing molecules and techniques can be used to convert codons encoding derived amino acid polymorphisms to codons encoding ancestral amino acid polymorphisms (e.g., including codons and polymorphisms set forth in Table 1 and SEQ ID NO: 1-21,318). Examples of such gene editing molecules include: (a) a nuclease comprising an RNA-guided nuclease, an RNA-guided DNA endonuclease or RNA directed DNA endonuclease (RdDe), a class 1 CRISPR type nuclease system, a class 2 type II Cas nuclease, a Cas9, a nCas9 nickase, a class 2 type V Cas nuclease, a Cas12a nuclease, a nCas12a nickase, a Cas12d (CasY), a Cas12e (CasX), a Cas12b (C2c1), a Cas12c (C2c3), a Cas12i, a Cas12j, a Cas14, an engineered nuclease, a codon-optimized nuclease, a zinc-finger nuclease (ZFN) or nickase, a transcription activator-like effector nuclease (TAL-effector nuclease or TALEN) or nickase (TALE-nickase), an Argonaute, and a meganuclease or engineered meganuclease; (b) a polynucleotide encoding one or more nucleases capable of effectuating site-specific alteration (including introduction of a DSB or SSB) of a target nucleotide sequence; (c) a guide RNA (gRNA) for use with an RNA-guided nuclease, or a DNA encoding a gRNA for use with an RNA-guided nuclease; (d) optionally donor DNA template polynucleotides suitable for insertion at a break in genomic DNA by homology-directed repair (HDR) or microhomology-mediated end joining (MMEJ); and (e) optionally other DNA templates (e.g., dsDNA, ssDNA, or combinations thereof) suitable for insertion at a break in genomic DNA (e.g., by non-homologous end joining (NHEJ). In certain embodiments, the at least one mutation is made with a cytosine and/or adenine base editor, or by a PRIME editing system. CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1. Plant RNA promoters for expressing CRISPR guide RNA and plant codon-optimized CRISPR Cas9 endonuclease are disclosed in International Patent Application PCT/US2015/018104 (published as WO 2015/131101 and claiming priority to U.S. Provisional Patent Application 61/945,700). Methods of using CRISPR technology for genome editing in plants are disclosed in US Patent Application Publications US 2015/0082478A1 and US 2015/0059010A1 and in International Patent Application PCT/US2015/038767 A1 (published as WO 2016/007347 and claiming priority to U.S. Provisional Patent Application 62/023,246). All of the patent publications referenced in this paragraph are incorporated herein by reference in their entirety. In certain embodiments, an RNA-guided endonuclease that leaves a blunt end following cleavage of the target site is used. Blunt-end cutting RNA-guided endonucleases include Cas9. In certain embodiments, an RNA-guided endonuclease that leaves a staggered single stranded DNA overhanging end following cleavage of the target site following cleavage of the target site is used. Staggered-end cutting RNA-guided endonucleases include Cas12a, Cas12b, Cas12d, Cas12e, and Cas12i.

Both adenosine nucleobase editors (e.g., Cas9 nickases operably linked to adenosine deaminases and suitable guide RNAs) and cytosine base editors (e.g., a dCas9 domain operably linked to a cytidine deaminase domain and a suitable guide RNAs) can also be used to convert codons encoding derived amino acid polymorphisms to codons encoding ancestral amino acid polymorphisms (e.g., including codons and polymorphisms set forth in Table 1 and SEQ ID NO: 1-21,318). Prime editing (PE) is a nucleic acid editing platform that enables the targeted and programmable installation of defined changes in a nucleotide sequence at a desired locus. It involves targeting of a prime editor to a target site in the genome, wherein the prime editor comprises a nucleic acid programmable DNA binding protein (napDNAbp) fused to a polymerase (e.g., a reverse transcriptase (RT)) associated with a prime editing guide RNA (pegRNA). The pegRNA comprises a scaffold (which binds to the napDNAbp, a spacer sequence (which is complementary to the genomic site), and an extension arm at the 3′ or 5′ end of the pegRNA. The extension arm includes a DNA synthesis template which includes the sequence of the desired edit. During prime editing, once the prime editor complexed with the pegRNA localizes to the genomic site, the polymerase (e.g., reverse transcriptase) synthesizes a new strand of DNA containing a desired edit using the DNA synthesis template. The new strand of DNA then replaces the corresponding endogenous DNA strand at the genomic site, thereby installing the desired, edited nucleotide sequence into the genome at the edit site. In some embodiments, techniques such as those taught in US PAT NOS. 10947530, 11214780B2, and U.S. Patent Application No. US20230357766A1 can be used for this purpose to convert at least one allele encoding a derived amino acid polymorphism of a plant genome to an allele encoding an ancestral amino acid polymorphism, and are hereby incorporated by reference in their entirety. Non-limiting examples of codons encoding derived amino acid polymorphisms include which can be converted to codons encoding ancestral amino acid polymorphisms include the codons and polymorphisms set forth in Table 1 and SEQ ID NO: 1-21,318.

### Breeding Strategies Using Identified Polymorphisms

1. Marker-Assisted Selection (MAS): Identified ancestral amino acid polymorphisms (e.g., ancestral amino acid polymorphisms set forth in Table 1 and SEQ ID NO: 1-21,318) and corresponding codon variants which encode the ancestral acid polymorphisms can serve as markers in marker-assisted selection to breed maize plants. By screening germplasm including seeds or seedlings for the presence of these markers (e.g., the presence of one or more ancestral amino acid polymorphisms in one or more orthologs), breeders can improve the agronomic fitness and/or accelerate the introduction of beneficial traits into new cultivars.

2. Genomic Selection: Genomic selection models incorporating ancestral amino acid polymorphisms (e.g., ancestral amino acid polymorphisms set forth in Table 1 and SEQ ID NO: 1-21,318) and corresponding codon variants which encode the ancestral acid polymorphisms can be used predict the breeding value of individual plants more accurately. This facilitates the selection of superior plants even before phenotypic traits are fully expressed.

The backcross breeding method provides a precise way of improving varieties that excel in a large number of attributes but are deficient in a few characteristics. (Page 150 of the Pr. R. W. Allard's 1960 book, published by John Wiley & Sons, Inc., Principles of Plant Breeding). The method makes use of a series of backcrosses to the variety to be improved during which the character or the characters in which improvement is sought is maintained by selection. At the end of the backcrossing the gene or genes being transferred unlike all other genes, will be heterozygous. Selfing after the last backcross produces homozygosity for this gene pair(s) and, coupled with selection, will result in a parental line of a hybrid variety with exactly or essentially the same adaptation, yielding ability and quality characteristics of the recurrent parent but superior to that parent in the particular characteristic(s) for which the improvement program was undertaken. Therefore, this method provides the plant breeder with a high degree of genetic control of his work.

In certain embodiments, methods of making a backcross conversion of maize plants with at least one ancestral amino acid polymorphism (e.g., ancestral amino acid polymorphisms set forth in Table 1 and SEQ ID NO: 1-21,318) are provided. In some embodiments, the method comprises crossing a maize inbred line plant comprising at least one ancestral amino acid polymorphism with a donor plant lacking the ancestral polymorphism(s) but further comprising one or more transgene(s), mutant gene(s), a naturally occurring gene(s) or a gene(s) and/or sequences modified through breeding techniques conferring one or more desired trait to produce F1 progeny plants. In some embodiments, the method further comprises selecting an F1 progeny plant comprising the ancestral polymorphism(s), transgene, naturally occurring gene(s), mutant gene(s) or modified gene(s) and/or sequences conferring the one or more desired trait. In some embodiments, the method further comprises backcrossing the selected progeny plant to the maize inbred line plants comprising the at least one ancestral amino acid polymorphism (i.e., the recurrent parent). This method can further comprise the steps of obtaining a molecular marker profile of a maize inbred line plant comprising at least one ancestral amino acid polymorphism and using the molecular marker profile to select for the progeny plant with the desired trait and the molecular marker profile of the parental maize inbred line plant. In certain embodiments, the maize inbred line plants comprising the at least one ancestral amino acid polymorphism (i.e., the recurrent parent) can comprise elite maize germplasm.

In certain embodiments, methods of making a backcross conversion of maize plants lacking at least one ancestral amino acid polymorphism (e.g., ancestral amino acid polymorphisms set forth in Table 1 and SEQ ID NO: 1-21,318) are provided. In some embodiments, the method comprises crossing a maize inbred line plant lacking the at least one ancestral amino acid polymorphism with a donor plant comprising the ancestral polymorphism(s) to produce F1 progeny plants. In some embodiments, the method further comprises selecting an F1 progeny plant comprising the ancestral polymorphism(s). In some embodiments, the method further comprises backcrossing the selected progeny plant to the maize inbred line plants lacking the at least one ancestral amino acid polymorphism (i.e., the recurrent parent) and selecting for progeny comprising the ancestral amino acid polymorphism. This method can further comprise the step of obtaining a molecular marker profile of a maize inbred line plant lacking at least one ancestral amino acid polymorphism and using the molecular marker profile to select for the progeny plant with the ancestral amino acid polymorphisms and the molecular marker profile of the parental maize inbred line plant. In certain embodiments, the maize inbred line plants lacking the at least one ancestral amino acid polymorphism(s) (i.e., the recurrent parent) can comprise elite maize germplasm.

In some embodiments, this method further comprises crossing the backcross progeny plant containing at least one ancestral amino acid polymorphism and the naturally occurring gene(s), the mutant gene(s) or the modified gene(s) and or sequences conferring the one or more desired trait with a second parental inbred maize line plant in order to produce a hybrid maize plant comprising the ancestral amino acid polymorphism, naturally occurring gene(s), the mutant gene(s) or modified gene(s) and/or sequences conferring the one or more desired traits. The plants or parts thereof produced by such methods are also part of the present disclosure. In certain embodiments, the second parental inbred maize line plant can comprise and/or be selected for the same ancestral amino acid polymorphism(s) of the progeny plant and/or can comprise and/or be selected for ancestral amino acid polymorphism(s) distinct from those of the progeny plant.

The disclosure further provides methods for developing maize plants in a maize plant breeding program using plant breeding techniques including but not limited to: recurrent selection, backcrossing, pedigree breeding, genomic selection, molecular marker (isozyme electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), and Simple Sequence Repeats (SSRs) which are also referred to as Microsatellites, Single Nucleotide Polymorphism (SNP), etc.) enhanced selection, genetic marker enhanced selection and transformation. Seeds, maize plants, and parts thereof produced by such breeding methods are also part of the disclosure.

Descriptions of other breeding methods that are commonly used for different traits and crops can be found in one of several reference books (e.g., R. W. Allard, 1960, Principles of Plant Breeding, John Wiley and Son, pp. 115-161; N. W. Simmonds, 1979, Principles of Crop Improvement, Longman Group Limited; W. R. Fehr, 1987, Principles of Crop Development, Macmillan Publishing Co.; N. F. Jensen, 1988, Plant Breeding Methodology, John Wiley & Sons).

In some embodiments of the disclosure, the number of ancestral amino acid polymorphisms that can be backcrossed into the maize inbred line is at least 1, 2, 3, 4, 5, or more. In certain embodiments, the number of ancestral amino acid polymorphisms that can be backcrossed into the maize inbred line is at least 10, 20, 50, 100, 200, 500, or more. In certain embodiments, at least 1, 2, 3, 4, 5, or more ancestral amino acid polymorphisms located in a single ortholog (e.g., a protein set forth in SEQ ID NO: 1-21.318) can be backcrossed into a maize inbred line. In certain embodiments, at least 1, 2, 3, 4, 5, or more ancestral amino acid polymorphisms located in a plurality of different orthologs (e.g., proteins set forth in SEQ ID NO: 1-21,318) can be backcrossed into a maize inbred line. In certain embodiments, the plurality of different orthologs (e.g., different proteins set forth in SEQ ID NO: 1-21,318) can comprise 2, 5, 10, 20, 50, 100, 200, or 500 different orthologs (e.g., proteins set forth in SEQ ID NO: 1-21.318).

### Maize Plant Cells, Plants, and Plant Parts

Maize plant cells, plants, and plant parts obtainable by the methods disclosed herein are provided. In certain embodiments, the maize plant cells, plants, or parts thereof comprise a substitution of one or more nucleotides in a codon of an endogenous gene encoding a derived amino acid polymorphism with one or more nucleotides to provide a codon variant encoding an ancestral amino acid polymorphism set forth in SEQ ID NO: 1-21,318, wherein one or more linked and/or unlinked DNA polymorphisms present in the maize plant cell comprising the codon variant are absent from maize plant cells comprising an endogenous maize gene which encodes the ancestral amino acid polymorphism. In certain embodiments, the maize plant cells, plants, or parts thereof comprise an insertion and/or deletion or substitution of one or more nucleotides in a codon of an endogenous gene encoding a derived amino acid polymorphism with one or more nucleotides to provide a codon variant encoding an ancestral amino acid polymorphism set forth in SEQ ID NO: 1-21,318, wherein one or more linked and/or unlinked DNA polymorphisms present in the maize plant cell comprising the codon variant are absent from maize plant cells comprising an endogenous maize gene which encodes the ancestral amino acid polymorphism. The chromosomal locations of the genes encoding the proteins set forth in SEQ ID NO: 1-21,318 are noted in Table 1 and are also available on the world wide web internet site “maizegdb.org;” Woodhouse et al., BMC Plant Biol 21, 385. doi: 10.1186/s12870-021-03173-5. In the aforementioned embodiments, linked markers are markers which are located on the same chromosome, same chromosome arm, or same chromosome interval. In the aforementioned embodiments, unlinked markers are markers which are located on different chromosomes. Suitable DNA polymorphisms include Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), and Simple Sequence Repeats (SSRs) which are also referred to as Microsatellites, and Single Nucleotide Polymorphism (SNP). Non-limiting examples of suitable SNPs which can be used as linked or unlinked DNA polymorphisms include maize SNPs set forth in US patent application publications US20060141495A1 and US20080083042A1, which are each incorporated herein by reference in their entireties. Maize germplasm comprising ancestral amino acid polymorphisms with linked and/or unlinked markers characteristic of maize germplasm lacking the ancestral amino acid polymorphisms is this provided.

### Embodiments

Various embodiments of the DNA molecules, plants, plant parts, genomes, chromosomes, methods, biological samples, and other compositions described herein are set forth in the following set of numbered embodiments.

1. A method for improving maize plant germplasm comprising:
(i) contacting the genomes of a plurality of maize plant cells comprising one or more codons encoding a derived amino acid polymorphism in at least one target maize gene with one or more gene editing reagents capable of converting said codons encoding the derived amino acid polymorphism to one or more codons encoding a corresponding ancestral amino acid polymorphism of the target maize gene; and
(ii) selecting a maize plant cell or plant wherein at least one codon encoding one or more derived amino acid polymorphisms of at least one target maize gene has been converted to the codon encoding the corresponding ancestral amino acid polymorphism of the target maize gene to obtain a first selected plant cell or plant containing the converted codon.

2. The method of embodiment 1, wherein the target maize gene encodes a protein comprising the amino acid sequence of SEQ ID NO: 1 to 21,318 or an allelic variant thereof wherein one or more of the variant residues of the protein is a derived amino acid polymorphism set forth in SEQ ID NO: 1 to 21,318.

3. The method of embodiment 1 or 2, wherein the corresponding ancestral amino acid polymorphism is an ancestral amino acid polymorphism set forth in SEQ ID NO: 1 to 21,318.

4. The method of any one of embodiments 1-31, wherein the target maize gene encodes a protein set forth in Table 1 or an allelic variant thereof, wherein the derived amino acid polymorphism comprises one or more of the derived amino acid polymorphisms set forth in Table 1, and wherein the ancestral amino acid polymorphism comprises the corresponding ancestral amino acid polymorphism set forth in Table 1.

5. The method of embodiment any one of embodiments 1-4, wherein the target maize gene encodes a protein set forth in Table 1 or an allelic variant thereof, wherein the derived amino acid polymorphism is encoded by a derived codon set forth in Table 1, and wherein the ancestral amino acid polymorphism is encoded by an ancestral codon set forth in Table 1.

6. The method of embodiment any one of embodiments 1-5, wherein at least 5, 10, 20, or 50 target maize genes are contacted with the gene-editing reagents and wherein the first selected maize plant cell or plant is selected for converted codons encoding the ancestral amino acid polymorphism in at least 5, 10, 20, or 50 of the target maize genes.

7. The method of any one of embodiments 1-6, wherein genomes of a plurality of plant cells or plants selected for converted codons encoding the ancestral amino acid polymorphism in at least 5 to 10 of the target maize genes are contacted with one or gene editing reagents capable of converting codons encoding the derived amino acid polymorphism to one or more codons encoding a corresponding ancestral amino acid polymorphism in at least 5 to 10 additional target maize genes.

8. The method of any one of embodiments 1-7, wherein the first selected plant is crossed to a second selected maize plant lacking the converted codon, producing a progeny maize plant comprising the converted codon and at least one additional marker characteristic of the second selected maize plant which is absent from the first selected plant.

9. The method of any one of embodiments 1-8, wherein the one or more codons encoding the derived amino acid polymorphism(s) are located on Chromosome 1, Chromosome 2, Chromosome 3 Chromosome 4, or Chromosome 5.

10. The method of embodiment any one of embodiments 1-9, wherein the one or more codons encoding the derived amino acid polymorphism(s) are located on Chromosome 6, Chromosome 7, Chromosome 8, Chromosome 9, or Chromosome 10.

11. The method of any one of embodiments 1-10, wherein the target maize gene comprising the ancestral amino acid polymorphism confers or contributes to improved agronomic fitness and/or an improvement in a desired agronomic trait in comparison to a control plant lacking the ancestral amino acid polymorphism(s).

12. The method of any one of embodiments 1-11, wherein the target maize gene confers or contributes to an improvement in photosynthesis, nitrogen utilization, nutrient uptake, or drought stability.

13. The method of any one of embodiments 1-12, wherein the target maize gene comprising the ancestral amino acid polymorphism confers or contributes to an improvement in plant stature, stalk diameter, plant height, vascular bundle density, vascular bundle area, or rind thickness in comparison to a control plant lacking the ancestral amino acid polymorphism(s).

14. The method of any one of embodiments 1-13, wherein the target maize gene comprising the ancestral amino acid polymorphism confers or contributes to improved nutritional quality in comparison to a control plant lacking the ancestral amino acid polymorphism(s).

15. The method of any one of embodiments 1-14, wherein the improved nutritional quality one or more of increased provitamin A content, increased lysine and/or tryptophan content, higher iron and zinc concentrations, reduced phytate content, higher amylopectin levels, increased oleic acid content, elevated anthocyanin levels, and/or improved dietary fiber content.

16. The method of any one of embodiments 1-15, wherein the target maize gene comprising the ancestral amino acid polymorphism confers or contributes to improved resistance to a pest in comparison to a control plant lacking the ancestral amino acid polymorphism(s).

17. The method of any one of embodiments 1-16, wherein the pest is an insect, a fungus, bacterium, or virus.

18. The method of any one of embodiments 1-17, wherein the target maize gene comprising the ancestral amino acid polymorphism confers or contributes to improved herbicide tolerance in comparison to a control plant lacking the ancestral amino acid polymorphism(s).

19. The method of any one of embodiments 1-18, wherein the herbicide is glyphosate, glufosinate, atrazine, metribuzin, bromoxynil, linuron, paraquat, lactofen, flumioxazin, carfentrazone, rimsulfuron, halosulfuron, chlorimuron, nicosulfuron, mesotrione, topramezone, tembotrione, isoxaflutole, pendimethalin, trifluralin, ethalfluralin, acetochlor, metolachlor, dimethenamid, 2,4-D, dicamba, or diflufenzopyr.

20. The method of any one of embodiments 1-18, wherein the target maize gene comprising the ancestral amino acid polymorphism confers or contributes to improved disease resistance in comparison to a control plant lacking the ancestral amino acid polymorphism(s).

21. The method of any one of embodiments 1-20, wherein the disease is northern leaf blight or southern leaf blight.

22. The method of any one of embodiments 1-21, wherein the target maize gene confers or contributes to an improvement in grain quality trait in comparison to a control plant lacking the ancestral amino acid polymorphism(s).

23. The method of any one of embodiments 1-22, wherein the grain quality trait is selected from the group consisting of starch digestibility, starch content, amylase content, kernel weight, and kernel hardness.

24. The method of any one of embodiments 1-23, wherein the protein comprising the derived amino acid polymorphism has decreased stability in comparison to the protein comprising the ancestral amino acid polymorphism.

25. The method of any one of embodiments 1-224 wherein the ancestral amino acid polymorphism encodes a protein with increased stability in comparison to the protein encoded by the derived amino acid polymorphism.

26. The method of any one of embodiments 1-25, further comprising obtaining the one or more gene editing reagents capable of converting said codon encoding the derived amino acid polymorphism(s) to a codon encoding the corresponding ancestral amino acid polymorphism of the target maize gene.

27. The method of any one of embodiments 1-26, wherein the gene editing reagent comprises a type II system (CRISPR-Cas9), or a type V system (CRISPR-Cas12a, or Cas12b, Cas12d, Cas12i, Cas12f), a cytosine base editor, or an adenine base editor system.

28. The method of any one of embodiments 1-26, wherein the gene editing reagent comprises a cytosine base editor, an adenine base editor, or a Prime editor.

29. The method of any one of embodiments 1-28, wherein the gene editing reagents comprise at least one guide RNA and at least one protein which recognizes the guide RNA or RNA encoding the protein, optionally wherein DNA encoding the guide RNA and protein are absent.

30. The method of any one of embodiments 1-29, wherein the one or more derived amino acid polymorphisms of at least one target maize gene have been converted to the corresponding ancestral amino acid polymorphism without introducing a foreign DNA sequence into the genome of the maize plant cell.

31. The method of any one of embodiments 1-30, further comprising molecular analysis to confirm the conversion of the derived amino acid polymorphism to the ancestral amino acid polymorphism.

32. The method of any one of embodiments 1-31, further comprising the step of regenerating a maize plant from the selected cell.

33. The method of any one of embodiments 1-31, wherein the target maize gene is selected from the group consisting of genes related to drought tolerance, nutrient use efficiency, or pest resistance.

34. A method for improving maize plant germplasm comprising:
(i) selecting a first parental maize plant comprising one or more codons encoding at least one desired ancestral amino acid polymorphism in one of more first parental plant gene(s) and a second parental maize plant comprising one or more codons encoding at least one desired ancestral amino acid polymorphism in one of more second parental plant gene(s);
(ii) crossing the first and second parental maize plants to obtain a population of progeny plants;
(iii) screening the population of progeny plants for a progeny plant comprising a combination of desired ancestral amino acid polymorphisms in both the first and second parental plant gene(s); and
(iv) isolating a progeny maize plant comprising the combination of desired ancestral amino acid polymorphisms from the population of maize plants.

35. The method of embodiment 34, further comprising selfing the isolated progeny maize plant and isolating a self (S1) progeny plant wherein at least one desired ancestral amino acid polymorphisms of both the first and second parental plant gene(s) is fixed in the S1 progeny plant.

36. The method of any one of embodiments 34 or 35, wherein the first and the second parental genes are each independently selected from genes encoding a protein comprising the amino acid sequence of SEQ ID NO: 1 to 21,318 or an allelic variant thereof wherein one or more of the variant residues of the protein is an ancestral amino acid polymorphism set forth in SEQ ID NO: 1 to 21,318.

37. The method of any one of embodiments 34-36, wherein the first and the second parental plant genes are each independently selected from genes encoding a protein set forth in Table 1 or an allelic variant thereof and wherein the ancestral amino acid polymorphism comprises the corresponding ancestral amino acid polymorphism set forth in Table 1.

38. A method for improving maize plant germplasm comprising:
(i) detecting a maize plant comprising a conversion of at least one codon encoding a derived amino acid polymorphism in at least one target gene to a codon encoding a corresponding ancestral amino acid polymorphism in a population of maize plants comprising individual maize plants comprising the codon encoding the derived amino acid polymorphism or the ancestral amino acid polymorphism; and
(ii) isolating the maize plant comprising the codon encoding the ancestral amino acid polymorphism from the population of maize plants.

39. The method of embodiment 38, wherein the conversion of the codon encoding the derived amino acid polymorphism is effected with a gene editing reagent.

40. The method of any one of embodiments 38 or 39, wherein the conversion is effected by crossing a first maize plant comprising the codon encoding the derived amino acid polymorphism with a second maize plant comprising the codon encoding the ancestral amino acid polymorphism to provide the population.

41. The method of any one of embodiments 38-40, wherein the detecting comprises identification of a maize plant in the population comprising the codon encoding the ancestral amino acid polymorphism of the second maize plant and a marker found in the first maize plant but not the second maize plant.

42. The method of any one of embodiments 38-41, further comprising producing a progeny plant from the cross wherein said progeny plant comprises at least one codon encoding the converted ancestral amino acid polymorphism.

43. The method of any one of embodiments 38-42 wherein the codon encoding the ancestral amino acid polymorphism confers or contributes to an improvement in agronomic fitness in comparison to a control maize plant lacking the codon encoding the ancestral amino acid polymorphism.

44. The method of any one of embodiments 38-43, wherein the improved agronomic fitness trait comprises drought tolerance, nutrient use efficiency, pest resistance, or increased yield.

45. The method of any one of embodiments 38-42 wherein the codon encoding the ancestral amino acid polymorphism confers or contributes to an improvement in nutritional quality in comparison to a control maize plant lacking the codon encoding the ancestral amino acid polymorphism.

46. The method of any one of embodiments 38-43, wherein the improved nutritional quality comprises one or more of increased provitamin A content, increased lysine and/or tryptophan content, higher iron and zinc concentrations, reduced phytate content, higher amylopectin levels, increased oleic acid content, elevated anthocyanin levels, and/or improved dietary fiber content.

47. The method of any one of embodiments 38-426 further comprising controlled pollination to enhance the likelihood of obtaining the maize plant germplasm with at least one converted ancestral amino acid polymorphism.

48. The method of any one of embodiments 38-47, wherein the selected maize plant comprises at least one ancestral amino acid polymorphism associated with increased protein stability as set forth in Table 1 and/or with increased protein function as compared to a protein having the derived allele.

49. The method of any one of embodiments 38-48, wherein the selected maize plant comprises at least one ancestral amino acid polymorphism associated with decreased protein stability as set forth in Table 1 and/or with decreased protein function as compared to a protein having the derived allele.

50. A method for identifying or selecting maize plants with improved fitness comprising: (i) identifying at least one ancestral amino acid polymorphism by performing genome-wide multiple sequence alignment between maize germplasm and related grass species, wherein the ancestral amino acid polymorphism comprises the allele found in at least 70% of the related grass species; (ii) identifying at least one derived amino acid polymorphism of the ancestral amino acid polymorphism, wherein a protein comprising the derived amino acid polymorphism has a predicted protein stability and/or a predicted protein function which is increased or decreased in comparison to a protein comprising the ancestral amino acid polymorphism; and optionally (iii) selecting a maize plant comprising the ancestral amino acid polymorphism from a population of maize plants comprising individual maize plants comprising the derived amino acid polymorphism or the ancestral amino acid polymorphism.

51. A method of identifying or selecting maize plants with improved agronomic fitness comprising:
(i) identifying at least one ancestral amino acid polymorphism by performing genome-wide multiple sequence alignment between maize germplasm and related grass species, wherein the ancestral amino acid polymorphism comprises the amino acid polymorphism found in at least 70% of the related grass species;
(ii) identifying at least one derived amino acid polymorphism of the ancestral amino acid polymorphism, wherein a protein comprising the derived amino acid polymorphism has a predicted protein stability and/or a predicted protein function which is increased or decreased in comparison to a protein comprising the ancestral amino acid polymorphism; and optionally
(iii) selecting a maize plant comprising the ancestral amino acid polymorphism from a population of maize plants comprising individual maize plants comprising the derived amino acid polymorphism or the ancestral amino acid polymorphism.

52. A method of identifying or selecting maize plants with improved nutritional quality comprising:
(i) identifying at least one ancestral amino acid polymorphism by performing genome-wide multiple sequence alignment between maize germplasm and related grass species, wherein the ancestral amino acid polymorphism comprises the amino acid polymorphism found in at least 70% of the related grass species;
(ii) identifying at least one derived amino acid polymorphism of the ancestral amino acid polymorphism, wherein a protein comprising the derived amino acid polymorphism has a predicted protein stability and/or a predicted protein function which is increased or decreased in comparison to a protein comprising the ancestral amino acid polymorphism; and optionally
(iii) selecting a maize plant comprising the ancestral amino acid polymorphism from a population of maize plants comprising individual maize plants comprising the derived amino acid polymorphism or the ancestral amino acid polymorphism.

53. The method of any one of embodiments 36 to 52, wherein the ancestral amino acid polymorphism is an ancestral amino acid polymorphism set forth in SEQ ID NO: 1 to 21,318.

54. The method of any one of embodiments 36 to 54, wherein the ancestral amino acid polymorphism is an ancestral amino acid polymorphism set forth in Table 1.

55. A method for producing a maize plant comprising in its genome at least one introgressed maize gene comprising a codon encoding an ancestral amino acid polymorphism, comprising the steps of:
(a) crossing a first maize plant with a second maize plant, wherein the second maize plant comprises an ancestral amino acid polymorphism, to obtain a population segregating for the ancestral amino acid polymorphism;
(b) selecting a seed or progeny maize plant from step (b) by its genotype, wherein the selected seed or progeny maize plant comprises a genotype comprising an ancestral amino acid polymorphism, thereby obtaining a selected maize plant comprising in its genome at least one introgressed maize gene comprising a codon encoding an ancestral amino acid polymorphism.

56. The method of embodiment 55, wherein said maize gene comprising the codon encoding said ancestral amino acid polymorphism is detected with a genotypic marker.

57. The method of any one of embodiments 55 or 56, wherein the codon encoding said ancestral amino acid polymorphism is detected by DNA sequencing.

58. The method of any one of embodiments 55-57 wherein said codon encoding said ancestral amino acid polymorphism is detected with a genotypic marker that is located within about 1000, 500, 100, 40, 20, 10, or 5 kilobases of said ancestral amino acid polymorphism.

59. A selected maize plant or part thereof produced by any one of embodiments 55-58 that comprises the introgressed maize gene comprising the codon encoding ancestral amino acid polymorphism.

60. The selected maize plant or part thereof of embodiment 59, wherein said introgressed ancestral amino acid polymorphism comprises the corresponding ancestral amino acid polymorphism of the derived amino acid polymorphism according to Table 1 and set forth in SEQ ID NO: 1 to 21,318, optionally wherein the part is a seed.

61. A maize plant cell comprising a substitution of one or more nucleotides in a codon of an endogenous gene encoding a derived amino acid polymorphism with one or more nucleotides to provide a codon variant encoding an ancestral amino acid polymorphism set forth in SEQ ID NO: 1-21,318, wherein one or more linked and/or unlinked DNA polymorphisms present in the maize plant cell comprising the codon variant are absent from maize plant cells comprising an endogenous maize gene which encodes the ancestral amino acid polymorphism.

62. A maize plant cell comprising an insertion and/or deletion or substitution of one or more nucleotides in a codon of an endogenous gene encoding a derived amino acid polymorphism with one or more nucleotides to provide a codon variant encoding an ancestral amino acid polymorphism set forth in SEQ ID NO: 1-21,318, wherein one or more linked and/or unlinked DNA polymorphisms present in the maize plant cell comprising the codon variant are absent from maize plant cells comprising an endogenous maize gene which encodes the ancestral amino acid polymorphism.

63. A maize plant part comprising the maize plant cell of any one of embodiments 60, 61, or 62, optionally wherein the part is a seed.

64. A maize plant cell, plant, or part thereof produced by the method of any one of embodiments 1-63.

### EXAMPLES

### Example 1. Inferring Ancestral and Derived Amino Acid Polymorphisms at Each Residue Predicted to Change Protein Stability

Whole-genome alignments in 19 grass species related to maize were performed and then used in cross species conservation analysis. Ancestral amino acid polymorphisms were defined as those that are most frequently observed at a given position in a multiple sequence alignment of multiple related species and thus likely to arise from a common ancestor and were assumed to be beneficial with the rationale that evolution is working to optimize protein function and will favor specific amino acids at a given residue. Amino acid polymorphisms with mutations that are rare occur more recently in a phylogeny and differ from the ancestral amino acid polymorphism and were assumed to be deleterious and defined as derived amino acid polymorphisms. Ancestral amino acid polymorphisms were inferred from a genome-wide multiple sequence alignment between maize (B73 v4) and 19 related grass species in the andropogonodea clade. Alignments were performed using the methods of Wu, Y., Johnson, L., Song, B., Romay, C., Stitzer, M., Siepel, A., Buckler, E., & Scheben, A. (2022), A multiple alignment workflow shows the effect of repeat masking and parameter tuning on alignment in plants. Plant Genome, 15, e20204. Ancestral amino acid polymorphisms were called for each position in the B74v4 maize genome where a reliable alignment could be obtained. Regions with fewer than 30% unambiguous amino acid polymorphsims calls (gaps in the alignment were considered as missing data). An amino acid polymorphism was considered to be ancestral if it was found in 70% or more of the species with an unambiguous amino acid polymorphism call at that position. Positions that did not meet these criteria were excluded from downstream analyses. To identify derived amino acid polymorphisms at each site, each of the ancestral amino acid polymorphisms identified above were compared with amino acid polymorphisms called from more than 1300 diverse maize inbreds. Any amino acid polymorphism that did not match the ancestral amino acid polymorphism was considered to be derived.

Ancestral and derived coding and protein sequences were constructed from the corresponding gene models from B73v4. First, the longest gene model for each gene in the B73v4 genome was selected. Next, it was determined whether B73 harbored the ancestral or derived amino acid polymorphism for each variant that overlapped with these regions. If the B73 amino acid polymorphism was ancestral, the sequence was translated in silico to generate an ancestral protein sequence, and the derived amino acid polymorphism was substituted, and the corresponding sequence was translated in silico to generate the derived protein sequence. Each protein sequence reported in Table 1 is the result of swapping one amino acid polymorphism at a time. Information set forth in the columns of Table 1 is further described in Table 2.

TABLE 2
Table 1 Column Descriptions

| SEQ ID NO | Unique Sequence ID |
|---|---|
| Protein ID | ID of the protein containing the mutation |
| Chromosome | Chromosome and Genomic position of the mutation in the B73v4 maize |
| Position | genomic map on the world wide web internet site maizegdb.org |
| SNP ID | Unique ID for the mutation |
| Protein Position | Position of the codon in the protein being affected by the mutation |
| Ancestral Codons | All codons which encode the ancestral amino acid |
| Derived Codons | All codons which encode the derived amino acid |
| Ancestral AA | Ancestral state (higher fitness) amino acid polymorphism |
| Derived AA | Derived state (lower fitness) amino acid polymorphism |
| Ancestral Stability | Protein stability for the ancestral state sequence |
| Derived Stability | Protein stability for the derived state sequence |
| Absolute Delta | Stability change between the ancestral and derived state protein |
| Stability | sequences |

To better identify conserved and functional residues, a trained natural language processing (NLP) model was fine-tuned on empirical protein stability metrics derived from yeast surface-display assay using synthetic proteins (Rocklin G J, Chidyausiku T M, Goreshnik I, Ford A, Houliston S, Lemak A, Carter L, Ravichandran R, Mulligan V K, Chevalier A, Arrowsmith C H, Baker D. Global analysis of protein folding using massively parallel design, synthesis, and testing. Science. 2017 Jul. 14; 357(6347): 168-175, which can be adapted for this purpose and is hereby incorporated by reference in its entirety.

This fine-tuned NLP model was used to predict the effects of naturally occurring missense and nonsense CDS variants that are segregating in cultivated maize on protein stability. This model takes a large population of extant genetic variants in maize and extracts variants that can impact biological functions through changes in protein stability. The model outputs a continuous metric for protein stability. Sites where at least two amino acid polymorphisms were found segregating in cultivated maize were predicted to significantly change protein stability (Table 1). This model gives insights into residues that are important for protein stability and amino acid polymorphisms that can change protein stability, but it cannot generalize across a diverse set of proteins which amino acid polymorphisms are beneficial. Amino acid polymorphisms that increase protein stability can be beneficial provided they do not result in a protein that is too rigid and cannot undergo the conformational changes necessary for optimal function. Alternatively, in certain instances decreased protein stability can be beneficial. To infer beneficial and deleterious amino acid polymorphisms at these residues, concepts from population and evolutionary genetics were leveraged.

### Example 2. Identifying a Set of Deleterious Amino Acid Polymorphisms Segregating in Cultivated Maize Through in Silico Validation

Weakly deleterious amino acid polymorphisms that change protein stability were predicted to have a significant, cumulative effect on hybrid performance (i.e. yield); thus, the total number of deleterious (i.e. derived) amino acid polymorphisms observed in a given hybrid should be inversely proportional to the performance of that hybrid. This hypothesis was tested by leveraging a large, genetically diverse collection of hybrids that were evaluated for yield in many locations throughout the US and had high-density genomic data that covered nearly all the putatively functional sites identified in the example 2. A linear mixed model framework was used to test whether the number of deleterious amino acid polymorphisms correlated with hybrid yield. In this model y is a vector of yield values for each maize hybrid, P is a matrix of the first four principal components from decomposition of a genomic relationship matrix (constructed according to VanRaden P M. Efficient methods to compute genomic predictions. J Dairy Sci. 2008 November; 91(11): 4414-23, hereby incorporated by reference for this purpose), x is a vector of coefficients for the principle components, z is a vector of genetic burden scores (the total number of deleterious amino acid polymorphisms observed in each hybrid genome) for each of the hybrids, and β is a coefficient that represents the average effect of a deleterious amino acid polymorphism on hybrid yield.

$$y = μ + x P + β z + e$$

The fined-tuned NLP model outputs a continuous metric for protein stability. Amino acid polymorphism sites were ordered by their predicted change in protein stability (highest to lowest) and defined a series of thresholds based on the quantiles of this distribution. For each threshold, the number of derived amino acid polymorphisms at sites that passed the threshold for each hybrid (genetic burden score) and fitted the model were counted. The threshold that showed the greatest association between hybrid yield and the number of deleterious (derived) amino acid polymorphisms was reported in Table 1. This analysis predicted that replacing 34 deleterious amino acid polymorphisms in a hybrid genome would improve yield by at least 4% over the unedited hybrid. Replacing 100 deleterious amino acid polymorphisms in a hybrid genome would improve yield by at least 12% over the unedited hybrid.

What is claimed is:

1. A method for improving maize plant germplasm comprising:
(i) contacting the genomes of a plurality of maize plant cells comprising one or more codons encoding a derived amino acid polymorphism in at least one target maize gene with one or more gene editing reagents capable of converting said codons encoding the derived amino acid polymorphism to one or more codons encoding a corresponding ancestral amino acid polymorphism of the target maize gene; and
(ii) selecting a maize plant cell or plant wherein at least one codon encoding one or more derived amino acid polymorphisms of at least one target maize gene has been converted to the codon encoding the corresponding ancestral amino acid polymorphism of the target maize gene to obtain a first selected plant cell or plant containing the converted codon.

2. The method of claim 1, wherein the target maize gene encodes a protein comprising the amino acid sequence of SEQ ID NO: 1 to 21,318 or an allelic variant thereof wherein one or more of the variant residues of the protein is a derived amino acid polymorphism set forth in SEQ ID NO: 1 to 21,318.

3. The method of claim 1, wherein the corresponding ancestral amino acid polymorphism is an ancestral amino acid polymorphism set forth in SEQ ID NO: 1 to 21,318.

4.-10. (canceled)

11. The method of claim 1, wherein the target maize gene comprising the ancestral amino acid polymorphism confers or contributes to improved agronomic fitness and/or an improvement in a desired agronomic trait in comparison to a control plant lacking the ancestral amino acid polymorphism(s).

12. (canceled)

13. The method of claim 1, wherein the target maize gene confers or contributes to an improvement in nutritional quality in comparison to a control plant lacking the ancestral amino acid polymorphism(s).

14.-17. (canceled)

18. The method of claim 1, wherein the target maize gene comprising the ancestral amino acid polymorphism confers or contributes to improved resistance to a pest in comparison to a control plant lacking the ancestral amino acid polymorphism(s).

19. (canceled)

20. The method of claim 1, wherein the target maize gene comprising the ancestral amino acid polymorphism confers or contributes to improved herbicide tolerance in comparison to a control plant lacking the ancestral amino acid polymorphism(s).

21. (canceled)

22. The method of claim 1, wherein the target maize gene comprising the ancestral amino acid polymorphism confers or contributes to improved disease resistance in comparison to a control plant lacking the ancestral amino acid polymorphism(s).

23.-25. (canceled)

26. The method of claim 1, wherein the protein comprising the derived amino acid polymorphism has decreased stability in comparison to the protein comprising the ancestral amino acid polymorphism.

27. (canceled)

28. The method of claim 1, further comprising obtaining the one or more gene editing reagents capable of converting said codon encoding the derived amino acid polymorphism(s) to a codon encoding the corresponding ancestral amino acid polymorphism of the target maize gene.

29.-31. (canceled)

32. The method of claim 1, wherein the one or more derived amino acid polymorphisms of at least one target maize gene have been converted to the corresponding ancestral amino acid polymorphism without introducing a foreign DNA sequence into the genome of the maize plant cell.

33.-35. (canceled)

36. A method for improving maize plant germplasm comprising:
(i) selecting a first parental maize plant comprising one or more codons encoding at least one desired ancestral amino acid polymorphism in one of more first parental plant gene(s) and a second parental maize plant comprising one or more codons encoding at least one desired ancestral amino acid polymorphism in one of more second parental plant gene(s);
(ii) crossing the first and second parental maize plants to obtain a population of progeny plants;
(iii) screening the population of progeny plants for a progeny plant comprising a combination of desired ancestral amino acid polymorphisms in both the first and second parental plant gene(s); and
(iv) isolating a progeny maize plant comprising the combination of desired ancestral amino acid polymorphisms from the population of maize plants.

37. (canceled)

38. The method of claim 36, wherein the first and the second parental genes are each independently selected from genes encoding a protein comprising the amino acid sequence of SEQ ID NO: 1 to 21,318 or an allelic variant thereof wherein one or more of the variant residues of the protein is an ancestral amino acid polymorphism set forth in SEQ ID NO: 1 to 21,318.

39.-65. (canceled)

66. A maize plant cell comprising an insertion and/or deletion or substitution of one or more nucleotides in a codon of an endogenous gene encoding a derived amino acid polymorphism with one or more nucleotides to provide a codon variant encoding an ancestral amino acid polymorphism set forth in SEQ ID NO: 1-21,318, wherein one or more linked and/or unlinked DNA polymorphisms present in the maize plant cell comprising the codon variant are absent from maize plant cells comprising an endogenous maize gene which encodes the ancestral amino acid polymorphism.

67.-68. (canceled)

69. The method of claim 36, wherein the progeny maize plant comprises in its genome at least one introgressed maize gene comprising a codon encoding the ancestral amino acid polymorphism.

70. The method of claim 36, further comprising detecting the progeny maize plant comprising the combination of desired ancestral amino acid polymorphisms in the population of progeny plants and isolating the detected progeny maize plant from the population.

71. The method of claim 1, wherein the ancestral amino acid polymorphism is identified by performing genome-wide multiple sequence alignment between maize germplasm and related grass species, wherein the ancestral amino acid polymorphism comprises the allele found in at least 70% of the related grass species.

72. A maize plant produced by the method of claim 36.

73. The method of claim 1, wherein the gene editing reagent comprises a CRISPR-Cas system, a cytosine base editor, an adenine base editor, or a prime editor.

74. A maize plant part comprising the maize plant cell of claim 66, optionally wherein the part is a seed.
